(19) United States
(12) Patent Application Publication
(10) Pub. No.: US 2007/0243258 A1
(43) Pub. Date: Oct. 18, 2007

(54) METHOD AND APPARATUS FOR FORMING CROSSLINKED CHROMONIC NANOPARTICLES

(75) Inventor: Eric R. Choban, St. Paul, MN (US)

Correspondence Address: 3M INNOVATIVE PROPERTIES COMPANY, PO BOX 33427, ST. PAUL, MN, 55133-3427, US

(73) Assignee: 3M Innovative Properties Company

(21) Appl. No.: 11/279,601

(22) Filed: Apr. 13, 2006

(51) Int. Cl. A61K 38/28; A61K 9/14

(52) U.S. Cl. 424/489; 514/3; 977/906; 422/261

(57) ABSTRACT

An apparatus and method of making crosslinked chromonic nanoparticles includes providing a first aqueous liquid stream including a continuous water-soluble polymer phase and a discontinuous chromonic material phase, providing a second aqueous liquid stream including a salt solution having a multivalent cation, and contacting the first aqueous liquid stream with the second aqueous liquid stream in parallel laminar flow to non-covalently crosslink the chromonic material with the multivalent cation, forming crosslinked chromonic nanoparticles. The chromonic material phase can optionally include an encapsulated guest molecule.

### BACKGROUND

The present disclosure relates to the field of chromonics. In particular, the present disclosure relates to a method and apparatus for forming crosslinked chromonic nanoparticles. The crosslinked chromonic nanoparticles can be used to encapsulate a guest molecule that can subsequently be released from the chromonic nanoparticles in a controlled manner.

Encapsulation and controlled release of a substance or material may be achieved by a number of methods. Typically, a polymeric coating may be used to either surround a substance or to form a mixture with a substance. Another common approach uses macroscopic structures having openings or membranes that allow for release of a substance. Encapsulation and controlled release find broad utility, but are particularly useful in the field of controlled release drug delivery.

Many polymeric coatings operate to control release by swelling in the presence of water. This relies on the mechanism of diffusion through a swollen matrix, which can be difficult to control. Alternatively polymeric coatings or mixtures of polymers with a substance may also operate through erosion or degradation of the polymer. In either case, it can be difficult to control the release rate, since most polymers are highly polydisperse in nature. In addition, there are a limited number of polymers suitable for use in pharmaceutical applications, and a given polymer may interact with different substances in very different and unpredictable ways.

Macroscopic structures, such as osmotic pumps, control release by uptake of water from the environment into a chamber containing a substance that is forced from the chamber through a delivery orifice. This, however, requires a complex structure that needs to be prepared and filled with the substance that is to be delivered.

Protection of a drug from adverse environmental conditions may be desirable in certain drug delivery applications. The gastrointestinal tract represents one example of an environment that can interfere with the therapeutic efficacy of a drug. The ability to selectively protect a drug from certain environmental conditions, such as the low pH of the stomach, and also to selectively and controllably deliver the drug under other environmental conditions, such as the neutral pH of the small intestine, is highly desirable.

In recent years, there also has been increasing research efforts to develop metal structures in the nanoscale range (that is, in the 0.1 to 100 nm range) for a variety of technological applications such as, for example, electronic and optical devices, labeling of biological material, magnetic recording media, and quantum computing.

### SUMMARY

The present disclosure provides methods and apparatus useful for forming crosslinked chromonic nanoparticles. In many embodiments, these crosslinked chromonic nanoparticles are useful for encapsulating and controlling the release of guest molecules, such as metals or bioactive compounds.

In one exemplary implementation, the present disclosure is directed to a method of making crosslinked chromonic nanoparticles. This method includes providing a first aqueous liquid stream containing a continuous water-soluble polymer phase and a discontinuous chromonic material phase, providing a second aqueous liquid stream containing a salt solution having a multivalent cation, and contacting the first aqueous liquid stream with the second aqueous liquid stream in parallel laminar flow to non-covalently crosslink the chromonic material with the multivalent cation, forming crosslinked chromonic nanoparticles.

In another exemplary implementation, the present disclosure is directed to an apparatus for making crosslinked chromonic nanoparticles. This apparatus includes an elongated laminar flow channel having a first end and an opposing second end, a first channel input adjacent the first end and in fluid communication with a source of a first aqueous liquid stream including a continuous water-soluble polymer phase and a discontinuous chromonic material phase, a second channel input adjacent the first end and in fluid communication with a source of a second aqueous liquid stream having a salt solution including a multivalent cation, and a channel output adjacent the second end and in fluid communication with a chromonic nanoparticle receiver.

These and other aspects of the apparatus and methods for making chromonic nanoparticles according to the subject invention will become readily apparent to those of ordinary skill in the art from the following detailed description together with the drawings.

### BRIEF DESCRIPTION OF THE DRAWINGS

So that those having ordinary skill in the art to which the subject invention pertains will more readily understand how to make and use the subject invention, exemplary embodiments thereof will be described in detail below with reference to the drawings, in which:

FIG. 1 is a schematic diagram of an illustrative apparatus for forming crosslinked chromonic nanoparticles; and

FIG. 2 is a schematic diagram of another illustrative apparatus for forming crosslinked chromonic nanoparticles.

### DETAILED DESCRIPTION

In view of the foregoing, it has been recognized that there is a need for a method for making crosslinked chromonic nanoparticles that provides control over the size and shape of the nanoparticles. Accordingly, the present disclosure provides a method and apparatus useful for forming crosslinked chromonic nanoparticles. In many embodiments, these crosslinked chromonic nanoparticles are useful for encapsulating a guest molecule such as a metal species or a bioactive compound. The guest molecule often can be released in a controlled manner. While the present invention is not so limited, an appreciation of various aspects of the invention will be gained through a discussion of the examples provided below.

The following description should be read with reference to the drawings, in which like elements in different drawings are numbered in like fashion. The drawings, which are not necessarily to scale, depict selected illustrative embodiments and are not intended to limit the scope of the disclosure. Although examples of construction, dimensions, and materials are illustrated for the various elements, those skilled in the art will recognize that many of the examples provided have suitable alternatives that may be utilized.

Unless otherwise indicated, all numbers expressing feature sizes, amounts, and physical properties used in the specification and claims are to be understood as being modified in all instances by the term “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein.

The recitation of numerical ranges by endpoints includes all numbers subsumed within that range (e.g. 1 to 5 includes 1, 1.5, 2, 2.75, 3, 3.80, 4, and 5) and any range within that range.

As used in this specification and the appended claims, the singular forms “a”, an” and “the” encompass embodiments having plural referents, unless the content clearly dictates otherwise. As used in this specification and the appended claims, the term “or” is generally employed in its sense including “and/or” unless the content clearly dictates otherwise.

As used herein, “nanoparticles” refers to particles of less than 1000 nanometers such as particles having a particle size in the range of 0.1 to 1000 nanometers or in the range of 0.1 to 100 nanometers.

The term “emulsion” refers to a discontinuous liquid phase that is distributed or suspended within a continuous liquid phase such that the discontinuous liquid phase does not coalesce over a useful time period (e.g., minutes, hours, or days).

The term “dispersion” refers to a discontinuous solid phase that is distributed or suspended within a continuous liquid phase such that the discontinuous solid phase does not coalesce over a useful time period (e.g., minutes, hours, or days).

The term, “chromonic materials” (or “chromonic compounds” or “chromonic molecules”) refers to large, multi-ring molecules typically characterized by the presence of a hydrophobic core surrounded by various hydrophilic groups (see, for example, Attwood, T. K., and Lydon, J. E., Molec. Crystals Liq. Crystals, 108, 349 (1984)). The hydrophobic core can contain aromatic and/or non-aromatic rings. When in solution, these chromonic materials tend to aggregate into a nematic ordering characterized by a long-range order.

The term “crosslinked chromonic nanoparticles” refers to a chromonic nanoparticles that is non-covalently crosslinked. The term “non-covalent crosslinking” refers to chromonic nanoparticles that are crosslinked without the formation of permanently formed covalent (or chemical) bonds. That is, the crosslinking does not result from a chemical reaction that leads to a new, larger molecule, but rather results from associations of multivalent cations with the chromonic molecules that are strong enough to hold them together without undergoing a chemical reaction. These interactions are typically ionic in nature and can result from interaction of a formal negative charge on the chromonic molecules with the formal positive charge of the multivalent cation. Since the multivalent cation has at least two positive charges, it is able to form an ionic bond with two or more chromonic molecules; that is, the multivalent cation forms a crosslink between two or more chromonic molecules. Divalent and/or trivalent cations are often preferred. In many embodiments, a majority of the multivalent cations are divalent. Suitable multivalent cations include any divalent or trivalent cations and include, but are not limited to, calcium, magnesium, zinc, aluminum, or iron.

The present disclosure is directed to a method of making crosslinked chromonic nanoparticles. This method includes providing a first aqueous liquid stream containing a continuous water-soluble polymer phase and a discontinuous chromonic material phase, providing a second aqueous liquid stream containing a salt solution having a multivalent cation, and contacting the first aqueous liquid stream with the second aqueous liquid stream in parallel laminar flow to non-covalently crosslink the chromonic material with the multivalent cation, forming crosslinked chromonic nanoparticles. The present invention is also directed to an apparatus suitable for making crosslinked chromonic nanoparticles.

Any chromonic material can be useful in the method of the invention. In many embodiments, the chromonic material or chromonic molecule is a non-polymeric molecule containing more than one carboxy functional group that can associate with monovalent or multivalent cations. The carboxy groups may be directly attached to an aromatic (e.g., carboxyphenyl) or heteroaromatic group. When the chromonic molecule has more than one aromatic or heteroaromatic group, the carboxy groups are arranged such that each aromatic or heteroaromatic group has no more than one carboxy group directly attached.

In some embodiments, the chromonic molecule contains at least one formal positive charge. For example, the chromonic molecule may be zwitterionic, that is, carrying at least one formal positive and at least one formal negative charge. In some chromonic molecules, the negative charge is carried by an acidic group having a dissociated hydrogen atom such as a carboxy group in its basic form, (i.e., —COO⁻). The negative charge can be carried by multiple carboxy functional groups, such that a proper representation of the chromonic molecule has two or more resonance structures, or structural isomers.

In many embodiments, chromonic materials include those represented by one of the following general structures of Formula I or Formula II.

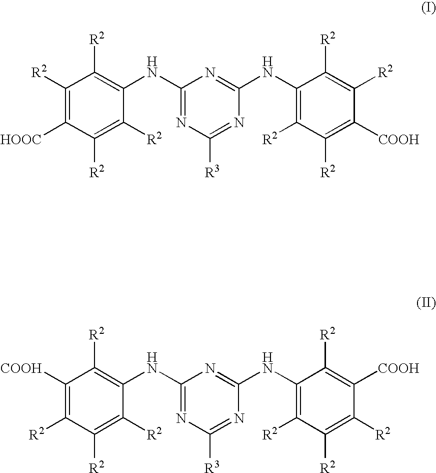

The compounds of Formula I have an orientation of the carboxy (—COOH) group that is para with respect to the amino linkage to the triazine center of the compound. The carboxy group may also be meta with respect to the amino linkage, as shown in Formula II (or may be a combination of para and meta orientations, which is not shown). In many compounds, the orientation is para. As depicted above, the chromonic molecules are neutral, but they may exist in alternative forms, such as a zwitterion or proton tautomer, for example where a hydrogen atom is dissociated from one of the carboxy groups and is associated with one of the nitrogen atoms in the triazine ring or with one of the amino linkages. The chromonic compound can also be a salt such as, for example, a carboxylate salt.

Each R² is independently an electron donating group, electron withdrawing group, or electron neutral group. In many embodiments, R² is hydrogen, a substituted or unsubstituted alkyl group, a substituted or unsubstituted alkoxy group (i.e., an alkoxy group is of formula —OR where R is an alkyl), or a substituted or unsubstituted carboxyalkyl group (i.e., a carboxyalkyl group is of formula —(CO)OR where (CO) denotes a carbonyl and R is an alkyl). Suitable substituents include hydroxy, alkoxy, carboxyalkyl, sulfonate, or halide groups. In some embodiments, R² is hydrogen.

Group R³ is a substituted heteroaromatic ring, unsubstituted heteroaromatic ring, substituted heterocyclic ring, or unsubstituted heterocyclic ring, that is linked to the triazine group through a nitrogen atom within the ring of R³. As used herein, a heteroaromatic ring refers to a heterocyclic ring that is fully conjugated. The R³ group can be, but is not limited to, heteroaromatic rings derived from pyridine, pyridazine, pyrimidine, pyrazine, imidazole, oxazole, isoxazole, thiazole, oxadiazole, thiadiazole, pyrazole, triazole, triazine, quinoline, or isoquinoline. In many embodiments, R³ includes a heteroaromatic ring derived from pyridine or imidazole. A substituent for the heteroaromatic ring R³ may be selected from, but is not limited to, any of the following substituted and unsubstituted groups: alkyl, carboxy, amino, alkoxy, thio, cyano, carbonylaminoalkyl (i.e., a group of formula —(CO)NHR where (CO) denotes a carbonyl and R is an alkyl), sulfonate, hydroxy, halide, perfluoroalkyl, aryl, alkoxy, or carboxyalkyl. In many embodiments, the substituent for R³ is selected from alkyl, sulfonate, carboxy, halide, perfluoroalkyl, aryl, alkoxy, or alkyl substituted with hydroxy, sulfonate, carboxy, halide, perfluoroalkyl, aryl, or alkoxy. In one embodiment, R³ is derived from a substituted pyridine with the substituent being preferably located at the 4-position. In another embodiment, R³ is derived from a substituted imidazole with the substituent being preferably located at the 3-position. Suitable examples of R³ include, but are not limited to: 4-(dimethylamino)pyridium-1-yl, 3-methylimidazolium-1-yl, 4-(pyrrolidin-1-yl)pyridium-1-yl, 4-isopropylpyridinium-1-yl, 4-[(2-hydroxyethyl)methylamino]pyridinium-1-yl, 4-(3-hydroxypropyl)pyridinium-1-yl, 4-methylpyridinium-1-yl, quinolinium-1-yl, 4-tert-butylpyridinium-1-yl, or 4-(2-sulfoethyl)pyridinium-1-yl, shown in Formulae IV to XIII below. Examples of heterocyclic rings that R³ may be selected from include, for example, morpholine, pyrrolidine, piperidine, or piperazine.

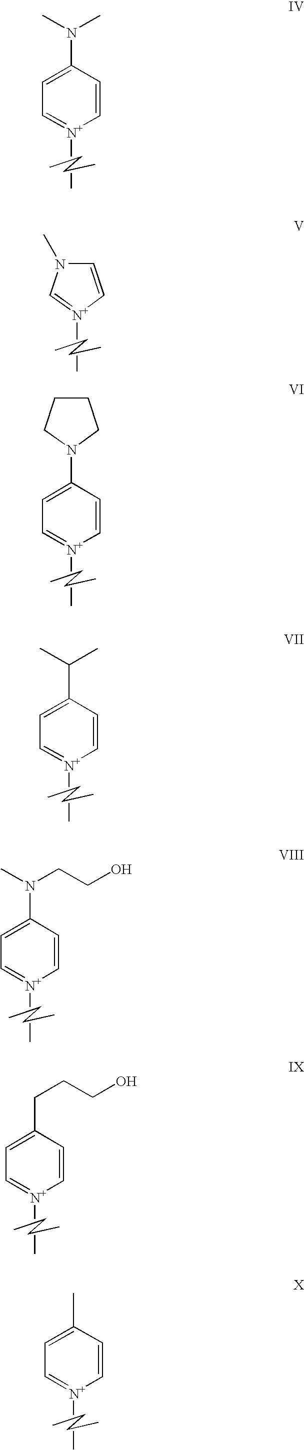

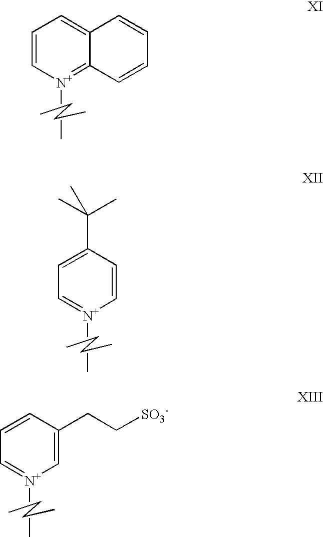

Some exemplary R³ groups are of Formula XIV 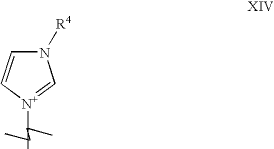
where R⁴ is hydrogen or a substituted or unsubstituted alkyl group. In many embodiments, R⁴ is hydrogen, an unsubstituted alkyl group, or an alkyl group substituted with a hydroxy, alkoxy, carboxyalkyl, sulfonate, or halide functional group. In some embodiments, R₄ is propyl sulfonic acid, methyl, or oleyl. Formula V is a subset of Formula XIV where R⁴ is methyl.

As depicted above the chromonic molecule of Formula I and II are neutral; however, chromonic molecules described herein may exist in an ionic form with at least one formal positive charge. In one embodiment, the chromonic molecule may be zwitterionic. An example of such a zwitterionic chromonic molecule, 4-({4-[(4-carboxylphenyl)amino]-6-[4-(dimethylamino)pyridinium-1-yl]-1,3,5-triazin-2-yl}amino)benzoate, is shown in Formula III below where R³ is a dimethylamino substituted pyridine ring linked to the triazine group through the nitrogen atom of the pyridine ring. As shown, the pyridine nitrogen carries a positive charge and one of the carboxy functional groups carries a negative charge (i.e., the carboxy group is in the dissociated basic form rather than acidic form). 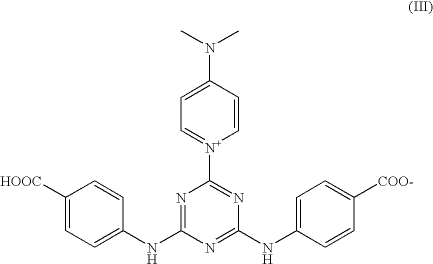
The chromonic molecule shown in Formula III may also exist in other tautomeric forms, such as where both carboxy functional groups carry a negative charge and where positive charges are carried by one of the nitrogen atoms in the triazine group and the nitrogen on the pyridine group.

In some embodiments, the chromonic compound can be represented by one of the following structures: 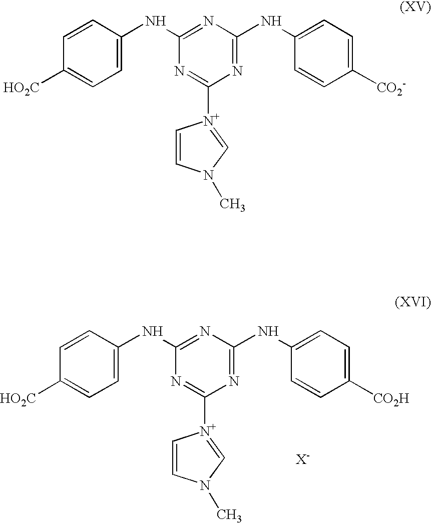
where X⁻ is a counterion. In some embodiments, X⁻ is selected from the group consisting of HSO₄⁻, Cl⁻, CH₃COO⁻, and CF₃COO⁻. Formula XV depicts the compound in its zwitterionic form. The imidazole nitrogen therefore carries a positive charge and one of the carboxy functional groups carries a negative charge (COO⁻). These compounds can also exist in other tautomeric forms such as where both carboxy functional groups carry a negative charge and where positive charges are carried by one of the nitrogen atoms in the triazine group and the nitrogen atom on the imidazole group.

As described in U.S. Pat. No. 5,948,487 (Sahouani et al.), which is herein incorporated by reference to the extent it does not conflict, triazine derivatives with formula I can be prepared as aqueous solutions. One synthetic route for the triazine molecules shown in formula I above involves a two-step process. Cyanuric chloride is treated with 4-aminobenzoic acid to give 4-{[4-(4-carboxyanilino)-6-chloro-1,3,5-triazin-2-yl]amino}benzoic acid. This intermediate is treated with a substituted or unsubstituted nitrogen-containing heterocycle. The nitrogen atom of the heterocycle displaces the chlorine atom on the triazine to form the corresponding chloride salt. The zwitterionic derivative, such as that shown in formula III above, is prepared by dissolving the chloride salt in ammonium hydroxide and passing it down an anion exchange column to replace the chloride with hydroxide, followed by solvent removal. Alternative structures, such as that shown in formula II above, may be obtained by using 3-aminobenzoic acid instead of 4-aminobenzoic acid.

Chromonic materials are capable of forming a chromonic phase or assembly. The chromonic phase can take on a number of morphologies, but is typically characterized by a tendency to form a stack of flat, multi-ring aromatic molecules. Ordered stacks of molecules are formed that grow with increasing concentration, but they are distinct from micellar phases, in that they generally do not have surfactant-like properties and do not exhibit a critical micellar concentration. In many embodiments, the chromonic phases will exhibit isodesmic behavior. That is, addition of molecules to the ordered stack leads to a monotonic decrease in free energy. The chromonic M phase (i.e., hexagonal phase) typically is characterized by ordered stacks of molecules arranged in a hexagonal lattice. The chromonic N phase (i.e., nematic phase) is characterized by a nematic array of columns, that is, there is long range ordering along the columns characteristic of a nematic phase, but there is little or no ordering amongst the columns, thus it is less ordered than the M phase. The chromonic N phase typically exhibits a schlieren texture, which is characterized by regions of varying index of refraction in a transparent medium.

The chromonic material is included in the first aqueous liquid stream. The first aqueous liquid stream includes a continuous water-soluble polymer phase and a discontinuous chromonic material phase. That is, the first aqueous liquid stream is an emulsion in which the chromonic material phase is distributed in the water-soluble polymer phase. The chromonic material is typically distributed as chromonic nanoparticles in an aqueous solution of the water-soluble polymer. This first aqueous liquid stream can be prepared, for example, by combining (a) an aqueous solution of a chromonic material and (b) an aqueous solution of a water-soluble polymer. Alternatively, the chromonic material can be dissolved in an aqueous solution and the water-soluble polymer in the form of a powder can then be dissolved in that aqueous solution.

The aqueous solution of the chromonic material can be prepared by dissolving the chromonic material in water that contains a base such as, for example, an amine, ammonium hydroxide, or an alkaline metal hydroxide (e.g., sodium hydroxide, potassium hydroxide, or lithium hydroxide). The pH is often at least 5, at least 6, at least 7, or at least 8. The aqueous solution can be heated, for example, to a temperature less than about 40° C. to facilitate dissolution of the chromonic material.

The aqueous solution of the chromonic material can optionally contain a surfactant. Suitable surfactants include ionic and non-ionic surfactants (preferably, non-ionic surfactants). An optional organic solvent (i.e., a water miscible organic solvent) such as a short chain alcohol can be added to the aqueous solution. The organic solvent can be added to the solution to achieve an organic solvent concentration in the range of 0.1 to 10 weight percent or in the range of 1 to 10 weight percent based on the weight of the aqueous solution.

In addition to the chromonic phase, the first aqueous liquid stream includes a continuous phase that contains a water-soluble polymer. The particular water-soluble polymer may influence the shape of the chromonic nanoparticles that are distributed within the continuous phase. Although not wanting to be bound by theory, the viscosity of the first aqueous liquid stream can influence the morphology of the chromonic phase distributed therein. In most instances, spherical chromonic nanoparticles are obtained. In some embodiments, acicular (needle-like) chromonic nanoparticles can result with the use of modified starch as the water-soluble polymer. The aspect ratios of these acicular chromonic nanoparticles can range from 1:4 to 1:10 and can have lengths from 300 nanometers to about 5 millimeters. In other embodiments, oblate spheroidal or toroidal shapes may be obtained.

In many embodiments, the water-soluble polymer often has a weight average molecular weight that is less than 2,000 g/mole, less than 5,000 g/mole, less than 10,000 g/mole, less than 20,000 g/mole, less than 50,000 g/mole, or less than 100,000 g/mole. Useful water-soluble polymers include, but are not limited to, polyvinyl-based water-soluble polymers, polycarboxylates, polyacrylates, polyamides, polyamines, polyglycols, cellulosics, starches and modified starches, or mixtures thereof. Copolymers such as block or random copolymers can also be useful. In some embodiments, the water-soluble polymers include, for example, cellulosics, starches (including modified starches such as phosphonated or sulfonated starches), polyvinyl alcohol, polyethylene glycol, polypropylene glycol, poly(ethylene glycol)-co-(propylene glycol), or mixtures thereof.

The relative concentrations of each of the components in the first aqueous liquid stream can vary with the desired size of the resulting crosslinked chromonics nanoparticles and their intended application. Generally, however, the chromonic material is added in an amount relative to the water-soluble polymer such that the chromonic phase is a discontinuous phase in a continuous phase that contains the water-soluble polymer. That is, chromonic nanoparticles are distributed in the water-soluble polymer continuous phase. The first aqueous liquid stream is an emulsion. The ratio of the water-soluble polymer to chromonic material is often at least 5:1 and less than 99:1, and preferably 3:1 to 15:1 on a dry weight basis. Generally, the water-soluble polymer is present in an amount in the range of 15 to 25 weight percent and the chromonic material is present in an amount in the range of 0.25 to 7 weight percent based on the weight of the first aqueous liquid stream.

The first aqueous liquid stream can contain other optional water-soluble molecules. For example, this liquid stream can include water-soluble molecules that enhance the homogeneity of the emulsion of chromonic nanoparticles within the water-soluble polymer. Suitable water-soluble molecules can include, but are not limited to, saccharides such as a monosaccharide (e.g., glucose or fructose), disaccharide (e.g., sucrose, maltose, or lactose), trisaccharide, or polysaccharide (e.g., starch, corn starch, amylopectin, maltodextrin, or corn syrup solids). The optional water soluble molecule can be present in any useful amount such as in an amount of 0 to 50 weight percent based on the weight of the first aqueous liquid stream.

Additionally, the first aqueous liquid stream can include an optional guest molecule. The guest molecule is usually encapsulated by the chromonic material (i.e., the guest molecule is encapsulated within the chromonic nanoparticles). That is, the chromonic nanoparticles distributed in the first aqueous liquid stream can contain guest molecules (i.e., the guest molecule is in the discontinuous chromonic phase). The guest molecule is often a metal salt, dye, cosmetic agent, fragrance, flavoring agent, or bioactive compound such as a drug, herbicide, pesticide, pheromone, or antifungal agent. The guest molecule is often mixed with the chromonic material prior to introduction of the water-soluble polymer.

Suitable metal salt for the guest molecules include silver salts (e.g., silver nitrate and silver acetate), gold salts (e.g., gold sodium thiomalate and gold chloride), platinum salts (e.g., platinum nitrate and platinum chloride), or mixtures thereof. In many embodiments, metal salts include, silver nitrate, silver acetate, gold sodium thiomalate, gold chloride, or mixtures thereof. Other transition metal salts may also be used such as salts of monovalent transition metal cations.

As used herein, the term “bioactive compound” refers to a compound that is intended for use in the diagnosis, cure, mitigation, treatment or prevention of disease, or to affect the structure or function of a living organism. In some embodiments, the bioactive compounds are drugs. Examples of suitable drugs include, but are not limited to, antiinflammatory drugs, both steroidal (e.g., hydrocortisone, prednisolone, and triamcinolone) and nonsteroidal (e.g., naproxen and piroxicam); systemic antibacterials (e.g., erythromycin, tetracycline, gentamycin, sulfathiazole, nitrofurantoin, vancomycin, penicillins such as penicillin V, cephalosporins such as cephalexin, and quinolones such as norfloxacin, flumequine, ciprofloxacin, and ibafloxacin); antiprotazoals (e.g., metronidazole); antifungals (e.g., nystatin); coronary vasodilators; calcium channel blockers (e.g., nifedipine and diltiazem); bronchodilators (e.g., theophylline, pirbuterol, salmeterol, and isoproterenol); enzyme inhibitors such as collagenase inhibitors, protease inhibitors, elastase inhibitors, lipoxygenase inhibitors, and angiotensin converting enzyme inhibitors (e.g., captopril and lisinopril); other antihypertensives (e.g., propranolol); leukotriene antagonists; anti-ulceratives such as H2 antagonists; steroidal hormones (e.g., progesterone, testosterone, and estradiol); local anesthetics (e.g., lidocaine, benzocaine, and propofol); cardiotonics (e.g., digitalis and digoxin); antitussives (e.g., codeine and dextromethorphan); antihistamines (e.g., diphenhydramine, chlorpheniramine, and terfenadine); narcotic analgesics (e.g., morphine and fentanyl); peptide hormones (e.g., human or animal growth hormones and LHRH); cardioactive products such as atriopeptides; proteinaceous products (e.g., insulin); enzymes (e.g., anti-plaque enzymes, lysozyme, and dextranase); antinauseants; anticonvulsants (e.g., carbamazine); immunosuppressives (e.g., cyclosporine); psychotherapeutics (e.g., diazepam); sedatives (e.g., phenobarbital); anticoagulants (e.g., heparin); analgesics (e.g., acetaminophen); antimigraine agents (e.g., ergotamine, melatonin, and sumatripan); antiarrhythmic agents (e.g., flecainide); antiemetics (e.g., metoclopromide and ondansetron); anticancer agents (e.g., methotrexate); neurologic agents such as anti-depressants (e.g., fluoxetine) and anti-anxiolytic drugs (e.g., paroxetine); hemostatics; and the like, as well as pharmaceutically acceptable salts and esters thereof. Proteins and peptides are particularly well suited guest molecules. Suitable examples include erythropoietins, interferons, insulin, monoclonal antibodies, blood factors, colony stimulating factors, growth hormones, interleukins, growth factors, therapeutic vaccines, and prophylactic vaccines. The amount of bioactive compound added to the first liquid stream is typically an amount that results in the encapsulation of about 0.1 to 70 weight percent bioactive compound based on the total weight of chromonic material and encapsulated bioactive compound.

A guest molecule, such as a drug, may be dissolved in an aqueous dispersant-containing solution prior to introduction of the chromonic molecules to the aqueous solution. Suitable dispersant include alkyl phosphates, phosphonates, sulfonates, sulfates, or carboxylates, including long chain saturated fatty acids or alcohols and mono or poly-unsaturated fatty acids or alcohols. Oleyl phosphonic acid is one example of a suitable dispersant. Although not to be bound by any particular theory, it is thought that the dispersant aids in dispersing the guest molecule so that it may be better encapsulated by the chromonic material.

The chromonic material and the guest molecule are often mixed together prior to introduction of the water-soluble polymer. The guest molecule is usually included within the chromonic phase in the first aqueous liquid stream. That is, the guest molecule is usually encapsulated within the chromonic nanoparticles and is present in the discontinuous chromonic phase within the first aqueous liquid stream. An alkaline compound may be added to the guest molecule solution prior to introduction of the chromonic material. Alternatively, an alkaline compound may be added to a chromonic molecule solution prior to mixing the guest molecule and chromonic molecule solutions. Examples of suitable alkaline compounds include ethanolamine, sodium or lithium hydroxide, or amines such as monoamines, diamines, triamines or polyamines. Although not to be bound by any particular theory, it is thought that alkaline compounds aid in dissolving the chromonic compound, particularly where the chromonic compound is a triazine compound such as those described in formulas I and II above.

This first aqueous liquid stream is contacted with a second aqueous liquid stream that contains a dissolved salt having a multivalent cation. The two streams are contacted in a parallel laminar flow arrangement. Although not wanting to be bound by theory, it is believed that the salt in the second aqueous liquid stream diffuses into the first aqueous liquid stream to non-covalently crosslink the chromonic nanoparticles. The first aqueous liquid stream often has an amber color while the second aqueous liquid stream often is clear and colorless. The contact of the first aqueous liquid stream with the second aqueous liquid stream results in the formation of a combined liquid stream that has a milky white appearance and that contains cross-linked chromonic nanoparticles. That is, crosslinked chromonic nanoparticles are dispersed in the combined liquid stream. The combined liquid stream often has a lower viscosity than the first aqueous liquid stream. The crosslinked chromonic nanoparticles (i.e., non-covalent crosslinked chromonic nanoparticles) have a reduced tendency to coalesce into larger particles compared to chromonic nanoparticles that are not crosslinked.

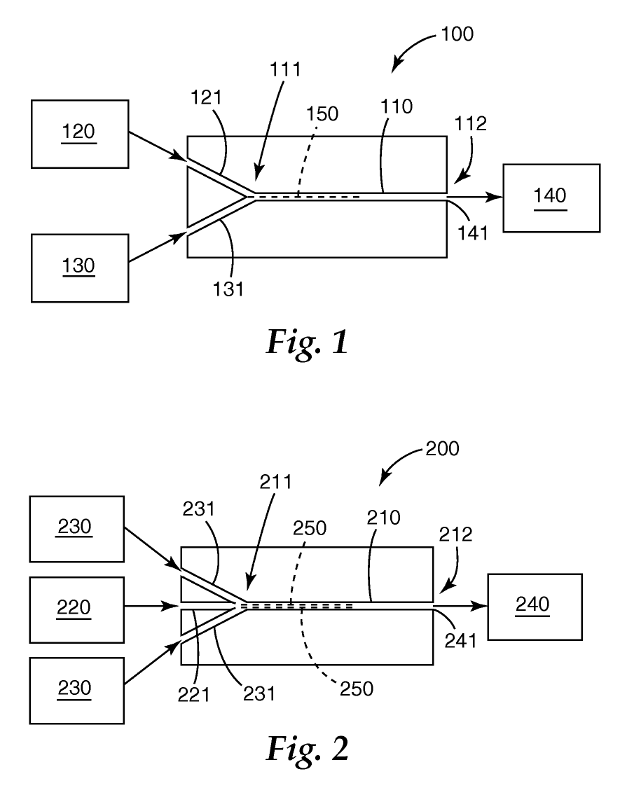

FIG. 1 is a schematic diagram of an illustrative apparatus 100 for forming the crosslinked chromonic nanoparticles that can optionally contain an encapsulated guest molecule. The apparatus 100 includes an elongated laminar flow channel 110 having a first end 111 and an opposing second end 112. A first channel input 121 is adjacent the first end 111 and in fluid communication with a source of a first aqueous liquid stream 120. The first aqueous liquid stream includes a continuous water-soluble polymer phase and a discontinuous chromonic material phase, as described above. This first aqueous liquid stream can also include an optional guest molecule within the chromonic phase. The guest molecule is typically encapsulated within the chromonic material of the chromonic nanoparticles. A second channel input 131 is adjacent the first end 111 and in fluid communication with a source of a second aqueous liquid stream 130. The second aqueous liquid stream 130 includes a salt solution having a multivalent cation, as described above. A channel output 141 is adjacent the second end 112 and in fluid communication with a chromonic nanoparticle receiver 140.

The first channel input 121 and the second channel input 131 are both in fluid communication with the laminar flow channel 1 10. The first aqueous liquid stream 120 and the second aqueous liquid stream 130 flow side-by-side in parallel laminar flow along the length of the elongated channel 110 and an interface 150 exists between the first aqueous liquid stream 120 and the second aqueous liquid stream 130. Diffusion between the first aqueous liquid stream 120 and the second aqueous liquid stream 130 occurs at the interface 150.

In some embodiments, the first liquid stream 120 can function as the feed for a plurality of first channel inputs 121 that is each, in turn, connected to a laminar flow channel 110 and a channel output 141. Likewise, in these embodiments, there can be a plurality of second channel inputs 131 that can be fed from a single second liquid stream 130 or from a plurality of second liquid streams 130. In other embodiments, the second liquid stream 130 can function as the feed for a plurality of second channel inputs 131 that are each, in turn, connected to a laminar flow channel 110 and channel output 141. Likewise, in these embodiments, there can be a plurality of first channel inputs 121 that can be fed from a single first liquid stream 120 or from a plurality of first liquid streams 120. Having a plurality of first liquid stream inputs 121, a plurality of second liquid stream inputs 131, a plurality of laminar flow channels 110, and a plurality of channel outputs 141 can increase the amount of crosslinked chromonic nanoparticles formed per unit of time.

FIG. 2 is a schematic diagram of another illustrative apparatus 200 for forming the crosslinked chromonic nanoparticles that can function as host materials. The apparatus 200 includes an elongated laminar flow channel 210 having a first end 211 and an opposing second end 212. A first channel input 221 is adjacent the first end 211 and in fluid communication with a source of a first aqueous liquid stream 220. The first aqueous liquid stream includes a continuous water-soluble polymer phase and a discontinuous chromonic material phase, as described herein. Other components as described above can also be included in the first aqueous liquid stream. Two second channel inputs 231 are adjacent the first end 211 and in fluid communication with a source of a second aqueous liquid stream 230. The second aqueous liquid stream 230 includes a salt solution having a multivalent cation, as described herein. The first channel input 221 is disposed between the two second channel inputs 231. A channel output 241 is adjacent the second end 212 and in fluid communication with a chromonic nanoparticle receiver 240.

The first liquid stream 220 in FIG. 2 can function as the feed for a plurality of first channel inputs 221 that is each, in turn, connected to a laminar flow channel 210 and a channel output 241. Likewise, in these embodiments, there can be a plurality of second channel inputs 231 that can be fed from a single second liquid stream 230 or from a plurality of second liquid streams 230. In other embodiments, the second liquid stream 230 can function as the feed for a plurality of second channel inputs 231 that are each, in turn, connected to a laminar flow channel 210 and channel output 241. Likewise, in these embodiments, there can be a plurality of first channel inputs 221 that can be fed from a single first liquid stream 220 or from a plurality of first liquid streams 220. Having a plurality of first channel inputs 221, a plurality of second channel inputs 231, a plurality of laminar flow channels 210, and a plurality of channel outputs 241 can increase the amount of crosslinked chromonic nanoparticles formed per unit of time.

The first aqueous liquid stream 220 and the second aqueous liquid stream 230 flow side-by-side in parallel laminar low along the length of the elongated channel 210 and an interface 250 exists between the first aqueous liquid stream 220 and the second aqueous liquid stream 230. Diffusion between the first aqueous liquid stream 220 and the second aqueous liquid stream 230 occurs at the interface 250.

The apparatus 100, 200 described herein can have any useful physical dimensions that allow for side-by-side parallel laminar flow of the two liquid input streams in the laminar flow channel 110, 210. The laminar flow channel often has a width in a range of 0.01 to 10 centimeters and, a depth in a range of 0.01 to 10 centimeters. The length of the laminar flow channel can be any suitable length. In some embodiments, the length of the laminar flow channel is at least 0.01 centimeters, at least 0.05 centimeters, at least 1 centimeter, at least 2 centimeters, or at least 5 centimeters. The length can be, for example, up to 100 meters, up to 50 meters, up to 20 meters, up to 10 meters, up to 1 meter, up to 500 centimeters, up to 200 centimeters, up to 100 centimeters, up to 50 centimeters, up to 20 centimeters, or up to 10 centimeters, In many embodiments, the length of the laminar flow channel is in the range of 0.01 centimeters up to 1 meter or in the range of 0.1 centimeter up to 100 centimeters. The illustrated liquid input streams and elongated channel have a “Y” configuration, however, the liquid input streams and elongated channel can have any useful configuration such as, for example a “T” configuration or any other configuration that allows for side-by-side parallel laminar flow of the two liquid input streams in the elongated laminar flow channel. The illustrated elongated laminar flow channel is shown having a linear length, however, elongated laminar flow channel can have any configuration that allows for side-by-side parallel laminar flow of the two liquid input streams in the laminar flow channel. In some embodiments, the elongated laminar flow channel has a non-linear configuration such as, for example, a “zig-zag” or serpentine configuration.

The first and second aqueous liquid streams are provided (often via a pump) to the elongated laminar flow channel at a flow rate that facilitates laminar flow along the length of the elongated laminar flow channel. Laminar flow occurs when two or more liquid streams having a certain characteristic (low Reynolds number) are joined into a single stream, also with a low Reynolds number, and as a result flow parallel to each other without turbulent mixing. The flow of liquids in capillaries often is laminar. The Reynolds number is a dimensionless fluid flow characteristic known to those of skill in the art and “laminar” flow is often associated with a Reynolds number of 2100 or less. In many embodiments, the Reynolds number of the first and second input streams and the side-by-side parallel laminar flow is at least 0.01, at least 0.05, at least 0.1, at least 0.2, at least 0.5, at least 1, at least 2, or at least 5. The Reynolds number is often no greater than 2000, no greater than 1500, no greater than 1000, no greater than 500, no greater than 200, or no greater than 100. For example, the Reynolds number can be in a range of 0.01 to 1000, in a range of 0.1 to 100, or in a range of 0.1 to 10.

Under conditions where laminar flow occurs, the multivalent cations in the second aqueous liquid stream diffuse into the first aqueous liquid stream containing chromonic nanoparticles. This diffusion results in the formation of non-covalently crosslinked chromonic nanoparticles. That is, non-covalent crosslinks are formed between chromonic materials in the chromonic nanoparticles. This method provides a continuous process for creating crosslinked chromonic nanoparticles. This method also has been found to reduce the time to crosslink chromonic nanoparticles in the first aqueous liquid stream verses a stagnant diffusion-based batch process by increasing the surface area to volume ratio in a continuous process. A turbulently mixed crosslinking batch process or turbulently mixed crosslinking continuous process usually results in dendritic like flocs or rods and not discrete particles and therefore is not a viable option for crosslinking of the chromonic nanoparticles.

The crosslinked chromonics nanoparticles that are part of the output liquid stream can be collected by, for example, filtration, spraying, or other means and dried to remove the continuous phase.

Subsequent to non-covalent crosslinking, the crosslinked chromonic nanoparticles may be contacted with a surface-modifying agent to render the nanoparticles more hydrophilic, hydrophobic, biocompatible, or bioactive, as desired. The surface groups are often present in an amount sufficient to form a monolayer, or a continuous monolayer, on the surface of the crosslinked chromonic nanoparticle.

Surface modifying groups may be derived from surface modifying agents. Schematically, surface modifying agents can be represented by the formula A-B, where the A group is capable of attaching to the surface of the crosslinked chromonic nanoparticle and the B group is a compatibilizing group that confers the desired hydrophilicity, hydrophobicity or biocompatibility. Compatibilizing groups can be selected to render the particle relatively more polar, relatively less polar or relatively non-polar.

Suitable classes of surface-modifying agents include organic oxyacids of carbon, sulfur and phosphorus, for example, alkylcarboxylates, alkyl sulfates, alkylsulfonates, alkyl phosphates and alkylphosphonates, glycoside phosphonates, salts thereof, and combinations thereof.

Representative examples of polar surface-modifying agents having carboxylic acid functionality include poly(ethylene glycol) monocarboxylic acid having the chemical structure CH₃O(CH₂CH₂O)ₙCH₂COOH or a salt thereof where n is in the range of 2 to 50 and 2-(2-methoxyethoxy)acetic acid or a salt thereof having the chemical structure CH₃OCH₂CH₂OCH₂COOH in either acid or salt forms.

Representative examples of non-polar surface-modifying agents having carboxylic acid functionality include octanoic acid, dodecanoic acid and oleic acid in either acid or salt form. In the case of a carboxylic acid containing olefinic unsaturation, such as oleic acid, the carbon-carbon double bonds may be present as either the Z or E stereoisomers or as a mixture thereof.

Examples of suitable phosphorus containing acids include alkylphosphonic acids including such as octylphosphonic acid, decylphosphonic acid, dodecylphosphonic acid, octadecylphosphonic acid, oleylphosphonic acid and poly(ethylene glycol) monophosphonic acid having the chemical structure CH₃O(CH₂CH₂O)ₙCH₂CH₂PO₃H₂ or a salt thereof where n is in the range of 2 to 50 in either acid or salt forms. In the case of a phosphonic acid containing olefinic unsaturation, such as oleylphosphonic acid, the carbon-carbon double bonds may be present as either the Z or E stereoisomers or as a mixture thereof.

Additional examples of suitable phosphorus containing acids include alkyl phosphates such as monoester and diesters of phosphoric acid including octyl phosphate, dodecyl phosphate, oleyl phosphate, dioleyl phosphate, oleyl methyl phosphate and poly(ethylene glycol) monophosphoric acid having the chemical structure CH₃O(CH₂CH₂O)ₙCH₂CH₂OPO₃H₂ or a salt thereof where n is in the range of 2 to 50.

In some modifications, the B group of the surface modifying agent A-B can also contain an additional specific functional group(s) to further adjust the hydrophilicity, hydrophobicity or biocompatibility of the chromonic nanoparticle. Suitable functional groups include, but are not limited to the hydroxyl, carbonyl, ester, amide, ether, amino, and quaternary ammonium functions.

If biocompatibility is desired, the chromonic nanoparticles may be surface modified with glycosides phosphonates, e.g. glucosides, mannosides, and galactosides of phosphonic acid.

In some embodiments, the crosslinked chromonics nanoparticles contain encapsulated metal ions (i.e., the crosslinked chromonics nanoparticles contain a guest metal ion). The metal ions can be reduced via reduction methods known in the art either before or after applying a dispersion containing the crosslinked chromonic nanoparticles to a surface of a substrate. The reduction can be accomplished by using a reducing agent (e.g., tris(dimethylamino)borane, sodium borohydride, potassium borohydride, or ammonium borohydride), electron beam (e-beam) processing, or ultraviolet (UV) light. After the metal ions are reduced, the coated layer can be dried and the chromonic material as well as the water-soluble polymer can be removed such that only metallic nanoparticles remain on the substrate as described above. The methods can be used to make spherical metallic nanoparticles that are substantially evenly spaced on a substrate surface. Drying of the coated layer can be achieved using any means suitable for drying aqueous coatings. Useful drying methods will not damage the coating or significantly disrupt the orientation of the coated layer imparted during coating or application.

The water-soluble polymer can be removed such that only the crosslinked chromonic nanoparticles (containing metallic or metal nanoparticles) remain on the substrate as discreet nanoparticles. For example, higher concentrations of water-soluble polymer tend to increase the spacing between crosslinked chromonic nanoparticles. Advantageously, unlike in other systems that phase separate (for example, polymer-polymer systems), the water-soluble polymer can be easily removed from the crosslinked chromonic nanoparticles. For example, the water-soluble polymer can be removed by heating to a temperature higher than the temperature at which the water-soluble polymer decomposes, but lower than which the chromonic material decomposes (for example, by heating to between about 200° C. and 350° C.).

The chromonic material can be removed using any means such as, for example by heating to decomposition (for example, by heating to higher than about 300° C.). Alternatively, if the substrate is glass, the chromonic material can be removed with a basic solution. Alternatively, the chromonic material can be rendered insoluble (for example, by protonization or amidization (that is, by reaction with diamine), or by thermally decomposing ammonium salts by heating to about 250° C.), and the water-soluble polymer can be removed with water.

The metallic chromonic nanoparticles may be used in such diverse applications as medical imaging, optical switching devices, optical communication systems, infrared detectors, infrared cloaking devices, chemical sensors, passive solar radiation collection or deflecting devices and the like.

Some crosslinked chromonic nanoparticles contain an encapsulated bioactive compound (i.e., the bioactive compound is a guest molecule). The encapsulated bioactive compound can subsequently be released in a controlled manner from the crosslinked chromonic nanoparticles. Drugs (i.e., pharmaceutically active ingredients), which are intended to have a therapeutic effect on an organism, are particularly useful guest molecules. Although any suitable type of drug may be encapsulated within the crosslinked chromonic nanoparticles, particularly suitable drugs include those that are relatively unstable when formulated as solid dosage forms, those that are adversely affected by the low pH conditions of the stomach, those that are adversely affected by exposure to enzymes in the gastrointestinal tract, and those that are desirable to provide to a patient via sustained or controlled release.

The crosslinked chromonic nanoparticles can selectively protect an encapsulated drug from certain environmental conditions and then controllably release the drug under other environmental conditions. When administered to an animal, the crosslinked chromonic nanoparticles are often stable in the acidic environment of the stomach but will release the guest molecule when passed into the non-acidic environment of the intestine (i.e. as result of a change in pH) or upon transport through the lumen of the small intestines and into the bloodstream. In some applications, the chromonic material encapsulating the drug can protect a drug from enzymatic degradation.

The crosslinked chromonic nanoparticles can often effectively isolate (i.e., encapsulate) drug molecules within the nanoparticles such that unfavorable interactions (e.g., chemical reactions) between different drugs in a combination dosage form, unfavorable changes in a single drug component (e.g., Ostwald ripening or particle growth, changes in crystalline form), and/or unfavorable interactions between a drug and one or more excipients can be avoided. In one example, the crosslinked chromonic nanoparticles allow two drugs that are ordinarily unstable in each other's presence to be formulated into a stable dosage form. In another example, the crosslinked chromonic nanoparticles allow a drug and excipient that are ordinarily unstable in each other's presence to be formulated into a stable dosage form.

Although large particles (e.g., on the order of several millimeters in diameter) may be prepared, the median diameters of the crosslinked chromonic nanoparticles are typically less than 1000 nanometers in size, usually less than 500 nanometers in size, and in some cases less than 100 nanometers. In some embodiments, the cross-linked chromonic particles have a mean diameter in the range of 10 to 1000 nanometers, in the range of 50 to 1000 nanometers, in the range of 100 to 1000 nanometers, in the range of 100 to 800 nanometers, in the range of 100 to 500 nanometers, or in the range of 100 to 400 nanometers. In certain instances it may be desired to have particles greater than 1 micrometer. These particle sizes may be desirable for oral delivery of drugs that are unstable in the intestine due to the presence of certain enzymes. Examples of such drugs include proteins, peptides, antibodies, and other biologic molecules that may be particularly sensitive to the body's enzymatic processes. In such cases, these small particles may be taken up into the intestinal wall directly, such that the particle primarily dissolves after passing the intestinal barrier, so that the amount of the sensitive drug exposed to the intestinal environment is minimized. Particles are typically spherical in their general shape, but may also take any other suitable shape, such as needles, cylinders, or plates.

The encapsulated guest molecule such as a drug can be released in an aqueous solution of univalent cations or other non-ionic compounds, such as surfactants. Typical univalent cations include sodium ions or potassium ions. The concentration of univalent cations needed to release the guest molecule will depend on the type and amount of the chromonic material included in the nanoparticles. For complete release of the guest molecule, there is often a molar excess of univalent cations compared to the molar amount of carboxyl groups in the chromonic material.

The rate of release of the guest molecule may also be controlled by adjusting the type and amount of multivalent cation used for crosslinking. Although divalent cations will be sufficient to crosslink the matrix, higher valency cations will provide additional crosslinking and lead to slower release rates. In addition to valency, release rate can also depend on the particular cation type. For example, a non-coordinating divalent cation (e.g., magnesium) will generally lead to faster release than a coordinating divalent cation (e.g., calcium or zinc) that has an empty electron orbital capable of forming a coordination bond with a free electron pair.

Different cation types as crosslinker of the chromonic material within the chromonic nanoparticles can be mixed so as to give an average cation valency that is not an integer. In particular, the use of a mixture of divalent and trivalent cations as the crosslinker can often result in a slower release of the guest molecule that rate than the use of all divalent cations. Although all of the guest molecules can be released over time, it may be desirable in certain applications to release only a portion of the guest molecules. The type or amount of chromonic material and multivalent cation can be adjusted such that the total amount of guest molecules that are released will vary depending on the environment into which they are placed. In some embodiments, the crosslinked chromonic nanoparticles will not release the guest molecule in an acidic solution, thus protecting acid sensitive guest molecules from degradation.

### EXAMPLES

All solvents, reagents and supplies to fabricate the fluidic device were obtained from Aldrich Chemical Company, Milwaukee, WI, unless otherwise noted.

All percents and amounts are by weight unless otherwise specified. As used herein, “HPMC” refers to hydroxypropylmethylcellulose having a number average molecular weight of approximately 10,000.

“Purified water” refers to water available under the trade designation “OMNISOLVE” from EMD Chemicals, Inc., Gibbstown, N.J.

A fluidic device consisting of a Y-channel with two inlets and one outlet was a stacked assembly consisting of 3 layers. The top and bottom layers were 15.9 cm (6.25 inches) long, 6.3 cm (2.5 inches) wide, 0.95 cm (0.375 inches) thick and made of polycarbonate (Lexan, available from Plastics International, Eden Prairie, Minn.). The middle layer was 15.9 cm (6.25 inches) long, 6.3 cm (2.50 inches) wide, and 0.38 cm (0.15 inches) thick, and made using Dow Corning Sylgard 183 (available from The Dow Chemical Co., Midland, Mich.). The bottom layer was machined to have a Y-channel with the 2 inlet sections of the “Y” that were of 2 cm (0.79 inches) long, 0.3 cm (0.12 inches) deep and 0.1 cm (0.0394 inches) wide with an angle of introduction of 45 degrees. The common channel, after the junction of the 2 inlet streams, was 10 cm (3.94 inches) long, 0.3 cm (0.12 inches) deep, and 0.1 cm (0.0394 inches) wide. The top layer functioned as a rigid capping layer. It also contained inlet and outlet holes with fitting assemblies (made of perfluoro-alkoxyalkane (PFA)). Tubing material made of polytetrafluoroethylene (PTFE) was used to transport fluid in and out of the device. The entire device was pressure sealed to eliminate leaks using bolts which held together all three layers. Eight bolts were located 3.8 cm (1.5 inch) apart from each other and 1.6 cm (0.625 inch) away from edges with the ninth bolt placed 1.3 cm (0.5 inch) away from the middle of the Y inlet.

Fluid flow through the device was pressure driven and controlled using a syringe pump (HA200OW/10 infuse/withdraw pump with a 6 to 10 rack, PHD2000 series, by Harvard Apparatus, of Holliston, Mass.) equipped with two different size syringes (Norm Ject 1.0 ml with 4.5 mm inner diameter (Syringe A) and Norm Ject 20.0 ml with 20.0 mm inner diameter (Syringe B), available from Henke Sass Wolf GMBH of Tuttlingen, Germany). Syringe A was used to pump the Chromonic/HPMC dispersion (i.e., the first aqueous liquid stream) and Syringe B was used with the salt solution (i.e., the second aqueous liquid stream. The flow rate was based on the 4.5 mm inner diameter syringe and was set to be 0.01 ml/min. This resulted in an overall average velocity of about 6.7 cm/min with a residence time of about 1.5 min and a Reynolds number of approximately 1 within the channel.

### Example 1

### Crosslinked Chromonic Nanoparticles

An aqueous solution of the chromonic compound of Formula XVI was prepared by stirring together the chromonic compound, (33% in water) and adding dropwise 1N NaOH to fully dissolve the chromonics material. A portion of this chromonics solution was then added to an aqueous solution of dissolved HPMC (25% solution). More specifically, the first aqueous liquid stream contained chromonic material (0.0638 g of a 33% solution) and HPMC (5.1 g of a 25% solution). This mixture was then stirred using a mechanical stirrer for 15 minutes before use.

The cross-linking solution was 10% ZnCl₂ in purified water (20 ml). After a processing period of 10 minutes using the fluidic device described herein, collected product was characterized using a particle size analyzer (Model ZEN3600, available from Malvern Instruments, Southborough, Mass.). The crosslinked chromonic nanoparticles had a mean particle size of approximately 398 nm.

### Example 2

### Crosslinked Chromonic Nanoparticles With Encapsulated Insulin

An aqueous solution of the chromonic compound of Formula XVI was prepared by stirring together the chromonic compound (33% in water), and adding dropwise 1N NaOH to fully dissolve the chromonics material. Hydrochloric acid (1N) in purified water was then added as needed to adjust the pH of the chromonic solution to ˜7.0. A solution of insulin from bovine pancreas was prepared by first mixing a portion of bovine insulin in purified water (1.65%) and a portion of aqueous oleyl phosphonic acid (3%) together followed by adjusting the pH of the resulting solution to ˜7.0 using either 1N Hydrochloric acid or aqueous NaOH (5%). A portion of this insulin mixture was added to the previously prepared chromonic mixture. A portion of this insulin/chromonic mixture was then added to an aqueous solution of HPMC (25%) and mechanically stirred for 15 minutes before use. More specifically, the first aqueous liquid stream contained chromonic material (29.8% aqueous solution, 2.5g) with insulin (44.2 mg of insulin/g of chromonic), andaqueous oleyl phosphonic acid (3% in purified water, 100 microliters) in the chromonic phase and HPMC (25% aqueous solution, 30.1g) as the continous water soluble phase

The cross-linking solution was 10% ZnCl₂ in purified water (20 ml). After a processing period of 10 minutes using the fluidic device described herein, collected product was characterized using a particle size analyzer (Model ZEN3600, available from Malvern Instruments, Southborough, Mass.). The crosslinked chromonic nanoparticles had a mean particle size of approximately 398 nm.

The present invention should not be considered limited to the particular examples described above, but rather should be understood to cover all aspects of the invention as fairly set out in the attached claims. Various modifications, equivalent processes, as well as numerous structures to which the present invention may be applicable will be readily apparent to those of skill in the art to which the present invention is directed upon review of the instant specification.

We claim:

1. A method of making crosslinked chromonic nanoparticles comprising:
providing a first aqueous liquid stream comprising a continuous water-soluble polymer phase and a discontinuous chromonic material phase;
providing a second aqueous liquid stream comprising a salt solution comprising a multivalent cation; and
contacting the first aqueous liquid stream with the second aqueous liquid stream in parallel laminar flow to non-covalently crosslink the chromonic material with the multivalent cation, forming crosslinked chromonic nanoparticles.

2. A method according to claim 1 wherein the contacting step comprises contacting the first aqueous liquid stream with the second aqueous liquid stream in parallel laminar flow within an elongated laminar flow channel to non-covalently crosslink the chromonic material with the multivalent cation, forming crosslinked chromonic nanoparticles.

3. A method according to claim 2 wherein the contacting step further comprises providing an elongated laminar flow channel having a first end and an opposing second end, and a first channel input adjacent the first end and in fluid communication with a source of the first aqueous liquid stream, a second channel input adjacent the first end and in fluid communication with a source of a second aqueous liquid stream, and a channel output adjacent the second end and in fluid communication with a crosslinked chromonic nanoparticle receiver.

4. A method according to claim 1 wherein the contacting step comprises contacting the first aqueous liquid stream with the second aqueous liquid stream in parallel laminar flow to non-covalently crosslink the chromonic material with the multivalent cation, forming crosslinked chromonic nanoparticles having a mean diameter in a range from 100 to 1000 nanometers.

5. A method according to claim 1 wherein the contacting step comprises contacting the first aqueous liquid stream with the second aqueous liquid stream in parallel laminar flow to non-covalently crosslink the chromonic material with the multivalent cation, forming crosslinked chromonic nanoparticles having an mean diameter in a range from 100 to 400 nanometers.

6. A method according to claim 1 wherein the providing a first aqueous liquid stream further comprises a drug.

7. A method according to claim 1 wherein the providing a first aqueous liquid stream further comprises a metal ion.

8. A method according to claim 1 wherein the providing a first aqueous liquid stream further comprises insulin.

9. A method according to claim 1 wherein the contacting step comprises contacting the first aqueous liquid stream with the second aqueous liquid stream in parallel laminar flow, having a Reynolds number in a range from 0.01 to 1000, within an elongated laminar flow channel to non-covalently crosslink the chromonic material with the multivalent cation, forming crosslinked chromonic nanoparticles.

10. An apparatus for making crosslinked chromonic nanoparticles comprising:
an elongated laminar flow channel having a first end and an opposing second end;
a first channel input adjacent the first end and in fluid communication with a source of a first aqueous liquid stream comprising a continuous water-soluble polymer phase and a discontinuous chromonic material phase;
a second channel input adjacent the first end and in fluid communication with a source of a second aqueous liquid stream comprising a salt solution comprising a multivalent cation; and
a channel output adjacent the second end and in fluid communication with a crosslinked chromonic nanoparticle receiver.

11. An apparatus according to claim 10, wherein the first channel input comprises two or more first channel inputs.

12. An apparatus according to claim 10, wherein the second channel input comprises two or more second channel inputs.

13. An apparatus according to claim 10, wherein the source of a first aqueous liquid stream further comprises a drug.

14. An apparatus according to claim 10, wherein the source of a first aqueous liquid stream further comprises a metal.

15. An apparatus according to claim 10, wherein the source of a first aqueous liquid stream further comprises insulin.

16. An apparatus according to claim 10, further comprising a first aqueous liquid stream pump in fluid communication with the source of a first aqueous liquid stream and the first channel input.

17. An apparatus according to claim 10, further comprising a second aqueous liquid stream pump in fluid communication with the source of a second aqueous liquid stream and the second channel input.

18. An apparatus according to claim 10, wherein the apparatus has a plurality of first channel inputs, a plurality of second channel inputs, a plurality of elongated flow channels, and a plurality of channel outputs.

19. An apparatus according to claim 18, wherein the plurality of first channel inputs are each in fluid communication with a common source of the first aqueous liquid stream.

20. An apparatus according to claim 18, wherein the plurality of second channel inputs are each in fluid communication with a common source of the second aqueous liquid stream.
